(19) United States
(12) Patent Application Publication
(10) Pub. No.: US 2002/0146441 A1
(43) Pub. Date: Oct. 10, 2002

(54) Selection method for cosmetic auxiliaries

(76) Inventors: Steffen Sonnenberg, Holzminden (DE); Anja Finke, Holzminden (DE); Simone Peters, Holzminden (DE); Andreas Klamt, Leverkusen (DE); John Lohrenz, Leverkusen (DE); Thorsten Burger, Koln (DE); Svend Matthiesen, Leverkusen (DE)

Correspondence Address: BAYER CORPORATION, PATENT DEPARTMENT, 100 BAYER ROAD, PITTSBURGH, PA, 15205, US

(21) Appl. No.: 10/029,725

(22) Filed: Dec. 20, 2001

(30) Foreign Application Priority Data
Dec. 27, 2000 (DE) 10065445.2

(51) Int. Cl. G06G 7/48; G06G 7/58; G06F 19/00; G01N 33/48; G01N 33/50; A61K 7/00

(52) U.S. Cl. 424/401; 702/19; 703/12

(57) ABSTRACT

A mathematical determination model is used to select cosmetic auxiliaries for cosmetic products and for the manufacture.

### FIELD OF THE INVENTION

The invention relates to a selection method for cosmetic auxiliaries for use in cosmetic products (cosmetics), in which mathematical determination models are used. The invention also relates to products to which cosmetic auxiliaries have been added, where the cosmetic auxiliaries are chosen using the mathematical determination models. The invention further relates to a selection method for the synthesis of novel cosmetic auxiliaries in which mathematical determination models are used. Furthermore, the invention relates to cosmetic auxiliaries for which mathematical determination models are used for selection for the preparation of cosmetic auxiliaries. The invention further relates to products to which cosmetic auxiliaries have been added, where mathematical determination models are used for the selection for the preparation of the cosmetic auxiliaries. The invention also relates to a method of predicting solubilities of cosmetic auxiliaries in cosmetic consumer products.

### BACKGROUND OF THE INVENTION

Cosmetic auxiliaries are used, for example, for improving the care, cooling, stabilizing, preserving and warming properties and for protecting against solar irradiation in numerous products (cosmetic consumer products). Through the addition of cosmetic auxiliaries, it is possible, for example, to intensify the care action of a skin cream, the cooling action of an aftersun lotion, the warming effect of an ointment and the sunscreen action of a sunscreen milk to a considerable extent. The use of cosmetic auxiliaries thus represents a product improvement.

Throughout all of the application steps of the various cosmetic consumer products, i.e. before, during and after application, part of the effect of the cosmetic auxiliaries is lost, meaning that their effect cannot be perceived by the user. Thus, for example, the formulations of various cosmetic consumer products may include some of the cosmetic auxiliaries in such a way that their action in the consumer product does not develop or is significantly reduced. Furthermore, transfer of the cosmetic auxiliaries within the scope of the application of a consumer product (cosmetic phase) to a substrate, such as, for example, textiles, skin or hair (phase to be cared for) is often incomplete as a consequence of specific interactions between the formulation of the consumer product and the substrate.

To solve this problem, on the basis of practical experience and after costly performance tests, the cosmetic auxiliaries chosen have hitherto been those which have the greatest effect in the cosmetic consumer product used. With these cosmetic auxiliaries, for example in the case of a sunscreen milk, the loss of chemical sunscreen filters during bathing is reduced, or for example in the case of a cooling ointment, the penetration of the skin by the cooling substance is improved. As a result, the overall effectiveness of the cosmetic auxiliary is increased accordingly. This method is very laborious and is unable to give a comprehensive overview with regard to the suitability of all relevant cosmetic auxiliaries in various formulations and all application steps of the product.

In addition, the preparation of stable cosmetic products is of interest. In the case of many cosmetic products, the auxiliaries used may lead to stability problems, e.g. as a result of precipitation or crystallization based on poor solubility of the cosmetic auxiliaries. In this regard, numerous experiments and experimental measurements as to the solubility of cosmetic auxiliaries in various products and formulations are carried out.

The formulation and preparation of cosmetic consumer products is generally known (A. Domsch, Die kosmetischen Präparate [Cosmetic preparations], 1994; K. Schrader, Grundlagen und Rezepturen der Kosmetika [Fundamentals and formulations of cosmetics], 1989; Harry's Cosmetology, 1973). The cosmetic auxiliaries for various consumer products are chosen by numerous performance tests in which, for example, the solubility of the individual cosmetic auxiliaries and the stability of the overall formulation is investigated.

The selection methods to date are often unsatisfactory since some of the cosmetic auxiliaries are unable to develop the desired effect because of specific interactions. Furthermore, because the solubility of the cosmetic auxiliaries is too low, unstable formulations may sometimes arise as a result, for example, of crystallization. In this regard, corresponding stability tests are carried out (K. Schrader, Grundlagen and Rezepturen der Kosmetika, 1989, p. 417).

The mode of action of cosmetic auxiliaries is known. Thus, for so-called cooling active ingredients, a connection between the penetration rate through the skin and the calcium ion concentration at the nerve endings in the skin is described (R. Pelzer, H&R Contact, 1198, p. 22-26). The mode of action of sunscreen filters is explained by N. Shaath (Sunscreens, Development, Evaluation, and Regulatory Aspects; 1997; The chemistry of sunscreens, p. 263-283). In the case of sunscreen filters, light is absorbed by molecules with conjugated double bonds in the wavelength range from 200-400 nm. A warming effect of so-called warming active ingredients can, for example, arise as a result of the heat of solution of e.g. glycols in water (ES 2,074,030; JP 06,080,534) or as a result of the direct influence of thermoreceptors by e.g. capsaicin (J. Szolcsanyi, F. Anton, P. Reeh, H. Handwerker; Brain Res. 1988, 446 (2), p. 262-268).

For the effectiveness of all cosmetic auxiliaries, a high concentration at the corresponding site of action is decisive. This means that a cosmetic auxiliary has to be transferred in a sufficient concentration onto the substrate, e.g. the skin, the hair or textiles, and must not remain permanently in the consumer product. In this connection, a partition parameter is defined as the distribution of the cosmetic auxiliary between the solid or liquid phase in the consumer product (cosmetic phase) or its application form, such as, for example, an aqueous solution, and the substrate (phase to be cared for): the higher the concentration of a cosmetic auxiliary on the substrate relative to the concentration of the cosmetic auxiliary in the solid or liquid phase of the consumer product, the higher the numerical value of the partition parameter. This distribution depends individually on the formulation of the consumer product and the application step in question, and on the specific molecular properties of the cosmetic auxiliary.

It is also known that different consumer products influence the action of cosmetic auxiliaries to markedly varying degrees (W. Johncock, Cosmetics & Toiletries, 1999, 9, Sunscreen interactions in formulations, p. 75-82). It is notable that even different formulations of a consumer product category, e.g. different skin cream, shampoo or soap formulations, differ in the transfer behavior of the cosmetic auxiliaries such that, for estimating the effectiveness, determination of the partition parameters should expediently be carried out for each individual formulation. In practice, this work cannot be carried out due to the enormous cost.

In addition, the influence of various emulsifiers on the sun protection factor (SPF) is described by G. Dahms, and a semiquantitative assessment is carried out (G. Dahms, Cosmetics & Toiletries, 1994, 11, Choosing emollients and emulsifier for sunscreen products, p. 45-52).

EP 0 386 898 describes the development of shampoo formulations, where a connection has been found between the amount of water-insoluble sunscreen filters which are transferred onto the hair during washing and the ingredients such as anionic washing-active substances, solvents and cationic derivatives of polygalactomannan gum.

In a QSAR (Böhm, Klebe, Kubinyi, Wirkstoffdesign [Active Ingredient Design], p. 363), a correlation between experimental values, such as, for example, the active concentration of active ingredients and physicochemical values is carried out. These physicochemical values, so-called descriptors, describe the chemical structure of the active ingredient. Within the cosmetics industry sector, the QSAR approach is used for explaining performance properties and for developing novel cosmetic auxiliaries. Thus, e.g. R. Peizer describes the development of novel cooling substances supported by Molecular Modelling (R. Pelzer, H&R Contact February 1998, P. 7-11).

In the field of material research, dielectric continuum models, such as, for example, COSMO (conductor-like screening model), PCM (polarizabie continuum model) and AMSOL are used as mathematical methods. In addition, COSMO-RS (conductor-like screening model for real solvents) is also used as a combination of COSMO with statistical thermodynamics. The semi-empirical determination model for the method according to the present invention has been publicized (J. Chem. Soc. Perkin Trans. 2 (1993) 799, J. Phys. Chem. 99 (1995), 2224, J. Phys. Chem. 102 (1998) 5074 and “COSMO and COSMO-RS” in “Encyclopedia of Computational Chemistry” Wiley Verlag New York (1998) and Fluid Phase Equilibria 172 (2000) 43).

The calculation method has been developed for calculating partition coefficients of organic molecules in ideal and real solvents which are present in a static partition equilibrium.

COSMO-RS has hereto been used for calculating physicochemical constants such as the boiling point, the vapor pressure or the partition equilibrium for octanol/water (logK_{OW}), hexane/water, benzene/water and diethyl ether/water (J. Phys. Chem. 102 (1998) 5074) and for calculating general liquid-liquid and liquid-vapor equilibria in process engineering.

Because the service life of cosmetic consumer products and of the cosmetic auxiliaries present therein is continually becoming shorter, an ever more rapid new development of formulations is necessary. The need for detailed investigations relating to the partition parameters and the solubilities of cosmetic auxiliaries thus increases as a function of the formulations of consumer products. Because of the large and still growing number of these investigations, it has for years been useful and desirable to develop a process for shortening these investigations. For this purpose, effective and reliable methods are necessary for predicting partition parameters and the solubilities of the cosmetic auxiliaries in different phases. These methods should permit the preparation of formulations which ensure a good solubility of the individual cosmetic auxiliaries, optimized release of the individual cosmetic auxiliaries from the formulation (cosmetic phase) at the desired point in time of application, and optimized transfer of the cosmetic auxiliaries to the desired substrate, such as, for example, textiles, skin or hair (phase to be cared for).

### SUMMARY OF THE INVENTION

We have found a method of selecting a cosmetic auxiliary or two or more cosmetic auxiliaries for a consumer product, which is characterized in that

in a first step for one group of cosmetic auxiliaries, a parameter is determined from the relative concentration of a cosmetic auxiliary in the phase to be cared for relative to the concentration in the cosmetic phase,

in a second step, the descriptors of cosmetic auxiliaries are determined using a mathematical method,

in a third step, the parameters determined in the first step are input into a determination model and a regression calculation is carried out,

in a fourth step, a prediction is made for all calculated cosmetic auxiliaries based on the regression calculation,

in a fifth step, the cosmetic auxiliaries most effective according to the prediction are used for the manufacture of the cosmetic product.

### DETAILED DESCRIPTION OF THE INVENTION

According to the method of the present invention, cosmetic auxiliaries are selected with a desired distribution between the cosmetic phase and the phase to be cared for, thus, for example, the optimum release of cosmetic auxiliaries from a consumer product or an optimized transfer of the cosmetic auxiliaries onto the desired substrate, such as e.g. textiles, skin or hair (phase to be cared for). This produces an optimum effectiveness during and after use of the consumer product. Furthermore, a more intensive and longer-lasting action arises, which can be perceived sensorily by the consumer.

At the same time, it is possible to minimize the amount of cosmetic auxiliaries as a function of the action to be achieved.

Surprisingly, using the mathematical determination models and the method according to the present invention, it is possible to calculate the partition parameters of cosmetic auxiliaries between a cosmetic phase and a phase to be cared for in dynamic and no longer only static systems comprising complex and nonuniformly structured phases, such as, for example, consumer products, and to predict them with outstanding accuracy. This means that although consumer products often consist of, for example, two or more nonideal phases and emulsions and although the determination models have been developed for calculating the static partition behavior of organic substances between two uniform solvents, it is surprisingly possible to make very good predictions for the partition behavior of cosmetic auxiliaries in consumer products. Furthermore, this method makes it possible to carry out the precise prediction of solubilities of cosmetic auxiliaries in cosmetic consumer products.

For the purposes of the present invention, cosmetic phases are liquid, solid and semisolid products which are to attain a care, cooling, stabilizing, preserving or protective action as a result of the addition of cosmetic auxiliaries. The cosmetic auxiliaries are transferred from these cosmetic phases into the phase to be cared for.

Furthermore, for the purposes of the present invention, cosmetic phases are in principle to be understood as meaning all natural or synthetic products which are changed as a result of the addition of cosmetic auxiliaries. The products to be cared for can be liquid or solid, but also semisolid (e.g. wax- or gel-like).

Preferred cosmetic products are, for example, use-specific consumer products for use as detergents, care compositions, air fresheners and cleaners for industrial application, in the domestic sector, for veterinary application and in body hygiene, and all application forms of the consumer products, such as, for example, aqueous solutions.

Preferred cosmetic products are, for example, shampoos, conditioners, hair colorants, deodorants, antiperspirants, solid and liquid soaps, body lotions, skin creams, washing powders, fabric softener compositions, fabric softener sheets, surface cleaners, toilet cleaners, rinses, all-purpose cleaners, disinfectants, polishes, glass cleaners, air fresheners, dishwashing compositions and waxes.

For the purposes of the present invention, phases to be cared for are liquid, solid and semisolid substrates which are to be cared for or are to attain a care property as a result of the transfer of the cosmetic auxiliaries from the cosmetic phase.

Preferred substrates which are of importance for everyday use by people are liquid phases to be cared for, such as, for example, aqueous solutions, and also solid surfaces to be cared for, such as, for example, textiles, skin, hair, plastics, metals, glass, ceramic, wood and stone.

Examples of cosmetic auxiliaries which can be added to the cosmetic are given, for example, in “Die kosmetischen Präparate”, “Grundlagen und Rezepturen der Kosmetika” and “Harry's Cosmetology” (A. Domsch, Die kosmetischen Präparate, 1994; K. Schrader, Grundlagen und Rezepturen der Kosmetika, 1989; Harry's Cosmetology, 1973).

Individual examples which may be mentioned are: sunscreen filters, such as, for example, p-aminobenzoic acid, ethyl p-aminobenzoate (25 mol) ethoxylated, 2-ethylhexyl p-dimethylaminobenzoate, ethyl p-aminobenzoate (2 mol) N-propoxylated, glycerol p-aminobenzoate, homomenthyl salicylate, 2-ethylhexyl salicylate, triethanolamine salicylate, 4-isopropylbenzyl salicylate, menthyl anthranilate, ethyl diisopropylcinnamate, 2-ethylhexyl p-methoxycinnamate, methyl diisopropylcinnamate, isoamyl p-methoxycinnamate, p-methoxycinnamic acid diethanolamine salt, isopropyl p-methoxycinnamate, 2-ethylhexyl 2-cyano-3,3-diphenyl acrylate, ethyl 2-cyano-3,3′-diphenyl acrylate, 2-phenylbenzimidazolesulphonic acid and salts and 3-(4′-trimethylammonium)-benzylidene-bornan-2-one methylsulfate, cooling substances, such as, for example, methoxypropanediols, methyidiisopropylpropionamide, menthyl PCA, ethylmenthanecarboxamides, menthone glycerol acetal, menthol, menthyl lactate and other menthol derivatives, warming substances, such as, for example, chili extract, 2-mercaptopyrimidine, capsaicin, capsaicin derivatives, glycols, polyalkylene glycols, isopropanol and polyalcohols, preservatives, such as, for example, benzoic acid, phenoxyethanol, ethylparaben, propylparaben, butylparaben, BHT and citric acid, antifoams, dyes, pigments which have a coloring action, thickeners, optical brighteners, moisturizers and/or humectants, fats, oils, waxes or other customary constituents of a cosmetic or dermatological formulation, such as climbazole, bisabolol and other alcohols, polyols, polymers, foam stabilizers, electrolytes, organic solvents or silicone derivatives.

In the method according to the present invention, in a first step, a parameter (partition equilibrium) is determined as a quotient from the relative concentration of the cosmetic auxiliary in the phase to be cared for and the cosmetic phase. Both the phase to be cared for and the cosmetic phase may be liquid, solid or semisolid. Preferably, the phase to be cared for is a liquid or solid phase.

It is preferred to determine the partition equilibrium between a liquid and a solid phase.

Alternatively, it is preferred to determine the partition equilibrium between two liquid phases.

Furthermore, it is preferred to determine the partition equilibrium between a liquid phase and a gas phase.

This distribution depends individually on the formulation of the consumer product and the respective application step and also on the specific molecular properties of the cosmetic auxiliaries. This product-specific parameter is the consequence of the specific interactions of the product or ingredients thereof with the individual cosmetic auxiliaries.

To determine the parameter, both the cosmetic consumer product, including all components such as the product itself, all cosmetic auxiliaries and other additives, and also simplified model products are taken into consideration.

Depending on the type of product, measurements of the cosmetic auxiliaries are made in the cosmetic product, in the individual application stages of the cosmetic product e.g. measurements in solutions, and on the various substrates to be cared for. For example, for a shampoo, the relative concentration of the cosmetic auxiliary such as e.g. sunscreen filter in the shampoo itself, is measured analytically, in a suitable aqueous solution, on the moist washed hair or on the dried hair.

For carrying out a regression calculation in the third step of the method according to the present invention, it is advantageous if 2 to 100 cosmetic auxiliaries are present as a group in the product to be investigated. It is preferred if approximately 5 to 50, and more preferred if 10 to 30, individual cosmetic auxiliaries are present in the cosmetic product to be investigated. This group of cosmetic auxiliaries, which should be structurally different, is representative of the totality of all cosmetic auxiliaries used for the manufacture of a certain consumer product. This group of cosmetic auxiliaries is incorporated into the product in a concentration customary for the type of product.

The relative concentration of the individual cosmetic auxiliaries is determined in a manner known per se by analytical methods, such as gas chromatography (GC), high performance liquid chromatography (HPLC), infrared spectrometry (IR), nuclear magnetic resonance spectrometry (NMR), mass spectrometry (MS) and ultraviolet spectrometry (UV). Furthermore, it is also possible to use signals of so-called electronic noses (D. Pybus, C. Sell, The Chemistry of Fragrances, p. 227-232). Gas chromatography and high performance liquid chromatography have proven particularly suitable for the analysis of cosmetic auxiliaries. In gas chromatography, it is also possible to use various injection methods, such as, for example, thermodesorption, liquid injection and gas injection.

Prior to analytical measurement of cosmetic auxiliaries, various enrichment processes can be carried out, such as, for example, extraction, concentration or adsorption. Suitable extractants or liquid-liquid or liquid-solid extractions are, for example, solvents, such as, for example, carbon dioxide, ethers, ketones, hydrocarbons, alcohols, water and esters.

Suitable for the adsorption or extraction of cosmetic auxiliaries from a product to be cared for are surface-active adsorbents, such as, for example, hair, textiles, ceramic, plastics, Tenax®, Poropax® and activated carbon. The cosmetic auxiliaries enriched on these adsorbents are then desorbed using heat (thermodesorption) or solvents and can then be analyzed.

In the second step, the descriptors of cosmetic auxiliaries are determined using a mathematical method. The descriptors describe properties such as, for example, the molecular weight, the molecular volume and the polarity.

In the first substep, conformers of the three-dimensional chemical structure of cosmetic auxiliaries to be calculated are generated using program such as, for example, Hiphop (Molecular Simulation Inc., USA) and HyperChem (Hypercube, Florida, USA).

(http://nhse.npac.syr.edu:8015/rib/repositories/csir/catalog/index.html)

A field of force optimization of the structures is then carried out using calculating programmes such as, for example, Discover (Insight, Molecular Simulation Inc., USA), Merck Molecular Force Field (MMFF, Merck) or Open Force Field (OFF, MSI, USA).

(http://nhse.npac.syr.edu:8015/rib/repositories/csir/catalog/index.html)

Subsequently, using accumulation analysis by means of cluster programmes such as, for example, NMRClust (Oxford Molecular Ltd, UK), those conformers are selected from the resulting molecular structures which have the greatest possible structural diversity.

(http://nhse.npac.syr.edu:8015/rib/repositories/csir/catalog/index.html). In particular, conformers with a low overall energy are preferred.

Subsequent structure optimization of the selected conformers is carried out using semiempirical calculation methods such as PM3 or AM1 (AMPAC, SemiChem or MOPAC, Fujitsu Ltd).

(http://nhse.npac.syr.edu:8015/rib/repositories/csir/catalog/index.html)

In a further accumulation analysis, the conformers are again selected for the further calculation.

(http://nhse.npac.syr.edu:8015/rib/repositories/csir/catalog/index.html)

Subsequently, a structure optimization and energy minimization is carried out using ab initio processes such as, for example, Hartree-Fock or Møller-Plesset or density functional methods (DFT) such as, for example, RI-DFT (Turbomol, Chem. Phys. Letters 162 (1989) 165) or GAUSSIAN98 (Gaussian Inc.) or DMol3 (Molecular Simulations Inc.) using the COSMO option.

(http://nhse.npac.syr.edu:8015/rib/repositories/csir/catalog/index.html)

A DFT/COSMO calculation gives, as a result, the total energy of the electrostatically ideally shielded molecule and the resulting shield charge density σ on the surface of the molecule.

In the subsequent step, COSMO-RS (COSMOlogic, Germany) is used to consider the interactions of molecules in liquid systems and amorphous solids as contact interactions of ideally shielded molecules (Fluid Phase Equilibria 172 (2000) 43).

In COSMO-RS calculations, the surface shield charge densities σ on the surface of a molecule of a substance X which are relevant for the interactions are in this case reduced to a frequency distribution p^{X}(σ), which gives the composition of the sections of surface with regard to σ and is abbreviated below to σ-profile.

Subsequently, the direct or the indirect calculation of the partition parameters can be carried out using two different methods. While for the direct calculation according to the known method (Fluid Phase Equilibria 172 (2000) 43), it is necessary to know the chemical composition of both phases (of the product and of the substrate), for the indirect calculation using the novel method, no information with regard to the chemical composition is necessary.

If the chemical composition of the two phases, such as, for example, in the case of wax and water, is known, it is possible to calculate the chemical potential of any compound in the phases directly using statistical thermodynamics. The logarithmic partition parameters then arise from the difference in the chemical potentials of the cosmetic auxiliary in the various phases.

In the rarest cases, the chemical and physical structure of the cosmetic consumer products is uniform and known to a degree such that it is possible to use the above-described method. In this case, a novel procedure is used in which the assumption is made that, as is the case for simple liquids, it is also possible to express the affinity for solvate molecules of very different polarity by a σ-potential μₛ(σ) for complex phases S, as are generally present in the case of consumer products to be cared for with cosmetic auxiliaries, if said potential can no longer be calculated directly using COSMO-RS. The shape of this function moves within the scope of the bandwidth of σ-potentials of organic liquids. For the calculation according to the present invention, μS(σ) is therefore expanded as a generalized Taylor series:

$${μ}_{S} ( σ ) ≅ {\sum }_{i = - 2}^{m} {c}_{S}^{i} {f}_{i} ( σ ) \qquad ( 1 )$$

where

ƒᵢ(σ)=σⁱ for i≧0 (2)

and

$${f}_{- 2 i - 1} ( σ ) = {f}_{acc / don} ( σ ) ≅ \{ \begin{matrix}0 & if & \pm σ < {σ}_{hb} \\ ∓ σ + {σ}_{hb} & if & \pm σ > {σ}_{hb}\end{matrix} \qquad ( 3 )$$

(Explanation of the symbols: μₛ(σ): σ-potential of the phase; i: index for counting the members of the series; m: highest order of the members of the series; fᵢ(σ): base function; acc: hydrogen bridge acceptor; cₛⁱ: expansion coefficient of the Taylor series; don: hydrogen bridge donor; σ_{hb}: threshold for hydrogen bridge bonds)

In the case of applications with equations, seven base functions suffice, i.e. the two hydrogen bridge functions f_{acc} (acceptor behavior), f_{don} (donor behavior) and the five polynomials Mᵢ^{X} of the order m=0 to m=4, in order to accommodate any σ-potentials for cosmetic auxiliaries sufficiently accurately by regression. The chemical potential of a substance X in this phase S can then be written as:

$${μ}_{S}^{X} = \int {p}^{X} ( σ ) {μ}_{S} ( σ ) ⅆ σ ≅ \int {p}^{X} ( σ ) {\sum }_{i = - 2}^{m} {c}_{S}^{i} {f}_{i} ( σ ) ⅆ σ ≅ {\sum }_{i = - 2}^{m} {c}_{S}^{i} {M}_{i}^{X} \qquad ( 4 )$$

where the σ-moments Mᵢ^{X} of the solvate are defined as

Mᵢ^{X}=∫p^{X}(σ)ƒᵢ(σ)dσ (5)

Using the seven σ-moments (f_{acc}, f_{don}, M₀^{X}, M₁^{X}, M₂^{X}, M₃^{X}, M₄^{X}) and μ_{gas}^{X}, a very generally valid principle of molecule descriptors has been found which makes it possible, according to equation (4), to determine any chemical potentials of cosmetic auxiliaries in very different matrices by linear regression. The phase S is characterized here by the coefficient cᵢ^{s} in front of the moments Mᵢ^{X}. In the case of charge-neutral substances, the first moment M₁^{X} is missing as descriptor since it describes the overall charge and assumes the numerical value zero. In the case of equilibria which involve the gas phase, the chemical potential μ_{gas}^{X} of the molecule in the gas phase is to be taken into consideration as descriptor in addition to the σ-moments. This is calculated directly by the COSMOtherm software.

In the third step of the method according to the invention, the parameters determined in the first step and the descriptors obtained in the second step, alone or in combination with already known descriptors, are input into the function equation of the mathematical determination model, and a regression calculation is carried out.

For this purpose, the measured relative concentrations of the individual cosmetic auxiliaries in the cosmetic phase and the phase to be cared for are compared. The partition parameter obtained for each individual cosmetic auxiliary is converted to the logarithm and used as so-called activity (Y) for a regression in a calculation table against the descriptors (X), and a regression calculation is carried out in a manner known per se (Böhm, Klebe, Kubinyi, Wirkstoffdesign [Active Ingredient Design], p. 370-372).

The above described σ-moment and μ_{gas}^{X} can be used, alone or in combination with already known descriptors, such as, for example, logP, both for the regression of partition parameters P^{X}_{gas,S} for substances X between a headspace under consideration and the cosmetic phase S, and also for the regression of partition parameters P^{X}_{S,S′} for substances X between a cosmetic phase S, e.g. an aqueous sunscreen product, and a phase S′ to be cared for, e.g. textiles or skin.

For the distribution of substances between the headspace under consideration and the cosmetic phase, if the setting approaches equilibrium, the logarithmic partition parameter P^{X}_{gas,S} is expressed as chemical potential difference in the determination model (6) below:

$$\begin{matrix}log {P}_{gas , S}^{X} = {c}_{gen} ( {μ}_{gas}^{X} - {μ}_{S}^{X} ) + const . \\ = {c}_{gen} {μ}_{gas}^{X} + {c}_{S}^{0} {M}_{0}^{X} + {c}_{S}^{2} {M}_{2}^{X} + {c}_{S}^{3} {M}_{3}^{X} + {c}_{S}^{4} {M}_{4}^{X} + \\ {c}_{S}^{acc} {M}_{acc}^{X} + {c}_{S}^{don} {M}_{don}^{X} + const .\end{matrix} \qquad ( 6 )$$

In the determination model (6), μ_{gas}^{X} is the chemical potential of the cosmetic auxiliary in the gas phase calculated directly using COSMO-RS. The coefficients cₛⁱ characterize the liquid or solid phase S with regard to their physical mode of interaction, while the general coefficient c_{gen} and the constant const. link together the system of units for free energies and logarithmic partition parameters. μ_{gas}^{X} and the above-defined moments Mᵢ^{X} are known from the COSMO-RS calculations.

If then the partition parameters for a group of 2 to 100 different cosmetic auxiliaries are known by analytical measurement, if the above-described COSMO-RS calculations are available, the missing coefficients for the descriptors are uniquely determined by linear regression.

For the partition parameter P^{X}_{S,S′}, which describes the distribution between a liquid or solid phase and between a liquid or solid phase, the gas phase potential μ_{gas}^{X} is insignificant. This then gives, analogous to equation (6):

$$\begin{matrix}log {P}_{S , {S}^{′}}^{X} = {c}_{gen} ( {μ}_{S}^{X} - {μ}_{{S}^{′}}^{X} ) + const . \\ = {c}_{S , {S}^{′}}^{0} {M}_{0}^{X} + {c}_{S , {S}^{′}}^{2} {M}_{2}^{X} + {c}_{S , {S}^{′}}^{3} {M}_{3}^{X} + {c}_{S , {S}^{′}}^{4} {M}_{4}^{X} + \\ {c}_{S , {S}^{′}}^{acc} {M}_{acc}^{X} + {c}_{S , {S}^{′}}^{don} {M}_{don}^{X} + const .\end{matrix} \qquad ( 7 )$$

Analogous to the partition parameter P^{X}_{gas,S}, reliable regressions with regard to the partition parameter P^{X}_{S,S′} are established by linear regression for any cosmetic auxiliaries for which the corresponding COSMO-RS calculations have been carried out.

Various regression methods, e.g. multiple linear regression, stepwise and GFA (genetic function algorithm), are used to ascertain equations which describe the mathematical relationship between the logarithmic partition parameters of the cosmetic auxiliaries and the descriptors. These equations are validated using various statistical methods, such as, for example, the correlation coefficient, standard deviation, chance test, number of degrees of freedom, number of outliers, boot strap error, cross validation, lack of fit (according to Jerome Friedman), determination of the deviations, F statistics, and other methods.

The quality of the mathematical relationship is better the closer the numerical values for the correlation coefficients r² and the cross validation XVr² come to the value 1, or the higher the numerical value for the F statistics (F test) and the lower the numerical values for the standard deviation s, outliers and lack of fit.

For the use of predictions for partition parameters of cosmetic auxiliaries, it is generally valid that the correlation coefficient r2 should be greater than 0.75 for a satisfactory correlation, greater than 0.85 for a good correlation and greater than 0.90 for a very good correlation. In order that a regression can be used for the prediction, the cross validation XVr² should be greater than 0.65 and preferably greater than 0.75 and not be more than 0.1 less than the associated correlation coefficient r².

The equation with the best correlation and best validation is used in order to calculate beforehand the logarithmic partition parameters in the determination model for all other cosmetic auxiliaries.

As a result of the regression calculation, in the fourth step of the method according to the present invention, exact predictions for the cosmetic auxiliaries under consideration with regard to the partition parameters between the cosmetic phase and the phase to be cared for are obtained as a result of inserting into equations (6) and (7) for all cosmetic auxiliaries, as a function of the coefficients and the descriptors. These predictions of the partition coefficients of the individual cosmetic auxiliaries are made available in data banks.

As a result of this prediction, in a fifth step for product development, individual or two or more cosmetic auxiliaries are selected which, based on the partition parameters, are particularly suitable for the manufacture of a desired product. Preference is given to cosmetic auxiliaries with the highest possible partition parameter or the highest possible transfer rate. These cosmetic auxiliaries are then used with other cosmetic auxiliaries in the development and manufacture of consumer products. The cosmetic auxiliaries chosen in this way are then added to the product in order to satisfy the expectations of the consumer of the product with regard to its care, cooling, stabilizing, preserving, sunprotecting and warming properties.

Using these cosmetic auxiliaries selected in this way, it is possible to create a consumer product with particularly good performance properties in one or more application stages for a consumer product.

Hereto, the mathematical description of partition parameters of cosmetic auxiliaries was possible only to an entirely inadequate degree and the selection of cosmetic auxiliaries in cosmetic products was carried out predominantly on the basis of empirical experiments and experience.

The advantage of the method according to the present invention lies in the universal and simple applicability of the calculation method for all partition parameters of cosmetic auxiliaries in any phases. The phases can have any desired composition which does not have to be known. The parameterization of the phases takes place via the coefficients of the descriptors in the regression equations. All descriptors are derived merely by calculation from the chemical structure of the cosmetic auxiliaries and do not require experimental work. Surprisingly, as a result of the method according to the present invention, an accurate and reliable mathematical description or explanation of the experimental partition parameters of cosmetic auxiliaries is possible. The accuracy and reliability compared with known methods and processes is thus, considerably improved. This means that by using the novel method, in contrast to existing methods, the first reliable prediction of partition parameters for cosmetic auxiliaries is possible. Furthermore, this method makes it possible to carry out the precise prediction of solubilities of cosmetic auxiliaries in cosmetic consumer products.

As a result, laborious experimental investigations into the partition parameters of cosmetic auxiliaries as a function of the formulation of a consumer product can be replaced by rapid, effective and reliable predictions. These predictions can be used to prepare particularly effective cosmetic products.

By using the mathematical method within the scope of the present invention, it is possible to carry out a reliable prediction of the partition parameters of cosmetic auxiliaries in various phases before, during and following application of consumer products.

As a result, it is possible to select cosmetic auxiliaries which have an optimal partition parameter for an existing formulation, for the preparation of cosmetic products. These consumer products have both an optimized action during use and also after use have a more intensive and longer-lasting action.

The invention also relates to products to which cosmetic auxiliaries have been added which are characterized in that the cosmetic auxiliaries are selected for the cosmetic products using a mathematical method. This method gives predictions with regard to the relative distribution of cosmetic auxiliaries in the phase to be cared for relative to the cosmetic phase.

The products according to the present invention to which cosmetic auxiliaries have been added are markedly superior in their use to cosmetics for which the cosmetic auxiliaries have been selected in a manner known per se.

The invention also relates to a selection process for the preparation of novel cosmetic auxiliaries, which is characterized in that mathematical determination models are used in the selection of the cosmetic auxiliaries to be prepared in a novel manner.

The novel cosmetic auxiliaries according to the present invention are markedly superior in their use to cosmetic auxiliaries which have been selected for the preparation in a manner known per se.

The present invention also relates to cosmetic auxiliaries which are characterized in that the selection for the preparation of the cosmetic auxiliaries is carried out using a mathematical determination model.

The novel cosmetic auxiliaries according to the present invention are markedly superior in their use to cosmetic auxiliaries which have been selected for the preparation in a manner known per se.

The present invention also relates to products to which cosmetic auxiliaries have been added, which are characterized in that the selection for the preparation of the cosmetic auxiliaries used for the cosmetic products is carried out using a mathematical method.

The products to which cosmetic auxiliaries have been added according to the present invention are markedly superior in their use to cosmetics for which the cosmetic auxiliaries have been selected for the preparation in a manner known per se.

The advantage of the method according to the present invention lies in the universal and simple applicability of the calculation method for all partition parameters of cosmetic auxiliaries in any phases. The phases can have any desired composition, which does not have to be known. The parameterization of these phases takes place via the coefficients of the descriptors in the regression equations. All descriptors are derived merely by calculation from the chemical structure of the cosmetic auxiliaries and do not require experimental work.

### EXAMPLES

In general, the analytical measurement of the relative concentration of cosmetic auxiliaries in a cosmetic product, in the headspace above the cosmetic product and on the substrate to be cared for are carried out, by way of example, for a group of cosmetic auxiliaries.

The cosmetic auxiliaries can be enriched using the various methods described above, and their concentrations can be measured. The enrichment method used in each case and the analytical measurement method are matched individually to the product to be measured and to the application step in each case.

The amounts of the cosmetic auxiliaries found in the phase to be cared for are compared with the amount found in the cosmetic phase (relative partition parameters). These values are converted to the logarithm and entered as activity values into the regression table (Table 1). Using various methods, regression against the COSMO-RS and other descriptors (e.g. clogP and boiling point) are carried out, and the best correlation according to the validation is selected. In all of the regression equations belonging to the examples, cosmetic auxiliaries with a deviation in the regression of more than +/−0.43 log units from the experimental value are defined as outliers. The COSMO-RS regression equations obtained in this way are significantly better compared with the clogP or b.p. regression equations with regard to the quality of correlation, quality of prediction and the number of outliers. In the next step, the COSMO-RS regression equation is linked to the regression table which contains all descriptors for all cosmetic auxiliaries. The use of the COSMO-RS regression equation on all cosmetic auxiliaries gives the prediction for the logarithmic relative partition parameters for all cosmetic auxiliaries. These values are then used for the manufacture of cosmetic products. The procedure is analogous in all examples.

The following chemical structure names are abbreviated: dimethylbenzylcarbinyl acetate (DMBCA), phenylethyl alcohol (PEA).

TABLE 1
Example of a regression table

| | Act- | | | | | | | |
|---|---|---|---|---|---|---|---|---|
| Substance | ivity | M₀^{x} | M₂^{x} | M₃^{x} | M₄^{x} | f_{acc} | f_{don} | ΔG_{Cosmo} |
| Neo Heliopan ® 303 | | 407.70 | 94.136 | 23.3745 | 63.5602 | 0.8874 | 0 | −29.6413 |
| Neo Heliopan ® 357 | | 355.96 | 97.522 | 17.0090 | 84.1261 | 1.7914 | 0.5442 | −26.5562 |
| Neo Heliopan ® AV | | 368.50 | 89.822 | 33.6316 | 83.7959 | 2.5385 | 0 | −25.2321 |
| Neo Heliopan ® BB | | 255.96 | 87.679 | 12.8657 | 79.2310 | 1.1989 | 0.7546 | −21.2202 |
| Neo Heliopan ® MDF | | 292.26 | 59.643 | 24.9599 | 47.7008 | 1.5264 | 0 | −21.2128 |
| Neo Heliopan ® HMS | | 299.77 | 60.154 | 10.0120 | 44.4878 | 0.3809 | 0.4555 | −19.4955 |
| Neo Heliopan ® Hydro | | 276.90 | 154.47 | −28.0202 | 263.8032 | 2.9901 | 7.8748 | −32.3986 |
| Neo Heliopan ® MA | | 316.45 | 75.055 | 5.9292 | 71.3452 | 1.0553 | 1.0306 | −22.2291 |
| Neo Heliopan ® OS | | 310.92 | 62.407 | 11.9810 | 49.1421 | 0.5393 | 0.4774 | −18.9196 |
| Neo Heliopan ® E1000 | | 315.38 | 85.783 | 31.3519 | 79.2203 | 2.2026 | 0 | −22.7417 |
| Neo Heliopan ® MBC | | 292.14 | 59.679 | 24.9716 | 47.7412 | 1.5283 | 0 | −21.2121 |
| alpha-Pinene | | 182.94 | 15.547 | 2.0076 | 3.7672 | 0.0036 | 0 | −9.6842 |
| Amylcinnamaldehyde, | | 270.30 | 60.582 | 22.1066 | 50.8991 | 1.4412 | 0 | −18.6148 |
| α | | | | | | | | |
| Benzyl acetate | | 197.88 | 72.379 | 27.692 | 64.7824 | 1.7606 | 0 | −16.4831 |
| Benzyl alcohol | | 150.59 | 65.602 | 6.9788 | 87.1685 | 2.333 | 1.6507 | −13.5795 |
| Benzyl salicylate | | 259.62 | 71.418 | 1.7256 | 47.1376 | 0.2492 | 0.4879 | −18.9653 |
| beta-Pinene | | 182.17 | 20.697 | 4.8988 | 7.2647 | 0.0087 | 0 | −10.232 |
| Camphene | | 179.45 | 19.913 | 4.4876 | 6.7674 | 0.0047 | 0 | −9.9874 |
| Caryophyllene | | 243.68 | 28.201 | 7.8143 | 11.5264 | 0.0747 | 0 | −14.1671 |
| Cedrol | | 253.34 | 47.908 | 15.6906 | 67.7804 | 2.3408 | 0.9288 | −16.476 |
| Cedryl acetate | | 279.10 | 50.828 | 28.4332 | 49.6005 | 1.7361 | 0 | −18.2928 |
| Citronellol | | 231.11 | 63.522 | 20.7436 | 94.9695 | 3.0534 | 1.5743 | −14.7972 |
| Coumarin | | 172.45 | 72.970 | 28.5131 | 82.5776 | 2.6523 | 0.001 | −18.9259 |
| Diethyl phthalate | | 250.88 | 88.235 | 40.9015 | 85.8762 | 2.0929 | 0 | −21.2179 |
| Dihydromyrcenol | | 227.10 | 56.430 | 24.3864 | 77.4421 | 2.8952 | 0.8139 | −14.0568 |
| DMBCA | | 235.19 | 64.333 | 26.0883 | 54.0729 | 1.5488 | 0 | −16.8222 |
| Ethylene brassylate | | 304.37 | 87.814 | 53.3295 | 94.7374 | 2.9648 | 0 | −24.9282 |
| Eugenol | | 211.09 | 71.061 | −2.7739 | 66.1703 | 0.4371 | 1.6307 | −16.2769 |
| gamma-Terpinene | | 196.66 | 24.722 | 6.1281 | 8.5462 | 0.0003 | 0 | −10.8848 |
| Geraniol | | 224.03 | 67.407 | 18.0173 | 96.9246 | 2.8774 | 1.7682 | −14.723 |
| Herbaflorat | | 224.20 | 59.936 | 33.5482 | 59.0415 | 1.7613 | 0 | −16.8667 |
| Hexylcinnamaldehyde, | | 286.74 | 62.029 | 23.913 | 51.3968 | 1.4679 | 0 | −19.5153 |
| α | | | | | | | | |
| Hydroxycitronellal | | 236.05 | 84.431 | 42.7048 | 121.5161 | 4.2171 | 1.1583 | −18.562 |
| Ionone, alpha | | 241.87 | 61.073 | 39.4863 | 70.3229 | 2.7962 | 0 | −18.1457 |
| Ionone, beta | | 244.28 | 57.905 | 39.5098 | 70.4258 | 3.0875 | 0 | −17.9204 |
| Iraldein, alpha | | 255.33 | 59.723 | 39.1916 | 68.4779 | 2.7242 | 0.0002 | −18.4167 |
| Isoamyl salicylate | | 255.85 | 62.035 | 12.2572 | 50.2951 | 0.6037 | 0.4704 | −16.697 |
| Isobornyl acetate | | 229.61 | 48.627 | 28.0637 | 49.6246 | 1.7189 | 0 | −15.726 |
| Lilial | | 261.24 | 63.727 | 24.457 | 48.0025 | 1.1775 | 0 | −19.1351 |
| Limonene D | | 196.12 | 27.540 | 8.2705 | 11.4017 | 0.0038 | 0 | −11.0721 |
| Linalool | | 221.83 | 59.129 | 20.8859 | 73.0534 | 2.4332 | 0.8688 | −13.7079 |
| Musk, ketone | | 288.19 | 84.911 | 22.3561 | 57.4895 | 0.6485 | 0 | −22.6713 |
| Musk, xylene | | 277.43 | 73.141 | 6.7028 | 35.7804 | 0 | 0 | −20.0232 |
| Oryclon | | 246.51 | 56.372 | 34.1065 | 58.5465 | 1.9711 | 0 | −16.6605 |
| Oryclon P 2 | | 243.70 | 52.932 | 32.4378 | 56.1438 | 2.0197 | 0 | −16.3314 |
| PEA | | 213.15 | 69.314 | 25.8393 | 56.9434 | 1.4132 | 0 | −16.4073 |
| Prenyl acetate | | 185.88 | 61.027 | 32.3413 | 61.077 | 1.8481 | 0 | −13.397 |
| Styrolyl acetate | | 211.06 | 66.887 | 26.2008 | 57.3712 | 1.5349 | 0 | −16.5608 |
| Terpineol | | 202.44 | 55.166 | 23.6513 | 79.1806 | 2.8931 | 0.9208 | −14.2052 |
| Terpinyl acetate | | 240.27 | 56.937 | 30.6541 | 53.5328 | 1.6327 | 0 | −16.2886 |

### Application Examples

### Example 1

O/W Emulsion, Substantivity on Skin (Water Resistance):

An exemplary formulation of a UV-protecting, perfumed O/W emulsion is as follows:

TABLE 2
O/W emulsion

| Part | Ingredients | | % (w/w) |
|---|---|---|---|
| A | Crodafos MCA (1) | Cetyl Phosphate | 1.50 |
| | Cutina MD (2) | Glyceryl Stearate | 2.00 |
| | Lanette 16 (2) | Cetyl Alcohol | 1.20 |
| | Cetiol SN (2) | Cetearyl Isononanoate | 8.00 |
| | Cetiol OE (2) | Dicaprylyl Ether | 7.00 |
| | Copherol 1250 (2) | Tocopheryl Acetate | 0.50 |
| | Solbrol P (3) | Propylparaben | 0.10 |
| | Neo Heliopan ® AV (4) | Ethylhexylmethoxy- | 1.00 |
| | | cinnamate | |
| | Neo Heliopan ® E1000 (4) | Isoamyl p- | 1.00 |
| | | Methoxycinnamate | |
| | Neo Heliopan ® OS (4) | 2-Ethylhexylsalicylate | 1.00 |
| | Neo Heliopan ® HMS (4) | Homosalate | 1.00 |
| | Neo Heliopan ® MA (4) | Menthyl anthranilate | 1.00 |
| | Neo Heliopan ® MBC (4) | 4-Methylbenzylidene | 1.00 |
| | | Camphor | |
| | Neo Heliopan ® BB (4) | Benzophenone-3 | 1.00 |
| | Neo Heliopan ® 357 (4) | | 1.00 |
| | Abil 100 (5) | Dimethicone | 0.30 |
| B | Water, dist. | Water (Aqua) | 62.30 |
| | 1,3-Butylene glycol | Butylene Glycol | 3.00 |
| | Solbrol M (3) | Methylparaben | 0.20 |
| | Phenoxyethanol (4) | Phenoxyethanol | 0.70 |
| | Carbopol ETD 2050 (6) | Carbomer | 0.20 |
| | Keltrol T (7) | Xanthan Gum | 0.20 |
| C | NaOH, 10% strength | Sodium Hydroxide | 2.80 |
| D | Fragrance (4) | Fragrance | 2.00 |

Suppliers:
(1) Croda Oleochemicals, cowick Hall Snaith Gool DN14 9AA, GB
(2) Cognis Deutschland GmbH, D-40191 Düsseldorf, Germany
(3) Bayer AG, Bayerwerk, D-51368 Leverkusen, Germany
(4) Haarmann & Reimer, D-37603 Holzminden, Germany
(5) Th. Goldschmidt AG, D-45127 Essen, Germany
(6) Goodrich Speciality Chemicals, OH 44141 Cleveland, USA
(7) Kelco International GmbH, CA 92123 San Diego, USA

The example reflects the substantivity of a sunscreen emulsion. The emulsion is applied to a skin model (sheep shorn wool, because of the content of wool grease), and the substantivity thereto is tested using a watering test. The emulsion is prepared in the generally customary manner. The UV filters and a mixture of 39 odorants are incorporated in accordance with the formulations. 0.10 g of this emulsion is applied to a section of the skin model 5×10 cm in size. After drying for 30 min, the sample is stirred for 20 min in 4.5 l of water at room temperature. The sample is left to dry for 12 hours and then boiled for 10 min with 60 ml of isopropanol in a closed system. The extract is transferred to a measuring flask and made up to 100 ml. 0.10 g of the emulsion is applied to another section of the skin model. After drying for 12 hours, it is extracted with isopropanol as above. The extracts are injected in equal amounts one after the other into a GC injector, and gas chromatograms are recorded. The amounts of the UV filters and odorants found in the extracts are compared with one another (relative partition parameters). The analytical results are then used mathematically as described above.

A correlation in accordance with the prior art leads to the results validated below:

Correlation with clogP as descriptor: r²=0.17, F Test=3.34, XVr²=−0.09, outliers: 0 of 18 substances.

Correlation with b.p. as descriptor: r²=0.01, F Test=0.19, XVr²=−0.61, outliers: 0 of 18 substances.

Correlation with b.p. and clogP as descriptors: r²=0.19, F Test=1.77, XVr²=−0.66, outliers: 0 of 18 substances.

COSMO-RS correlation: r²=0.75, F Test=9.17, XVr²=0.28, with descriptors: M₂^{X}, M₃^{X}, f_{acc}, f_{don}, outliers: 0 of 18 substances.

TABLE 3
Example logarithmic partition parameters for substantivity on
a skin model

| Substance | Activity | Prediction | Differences |
|---|---|---|---|
| Neo Heliopan ® MBC | −0.0966 | −0.1390 | 0.0424 |
| Neo Heliopan ® E1000 | −0.0762 | −0.1046 | 0.0284 |
| Neo Heliopan ® OS | −0.1464 | −0.1231 | −0.0233 |
| Neo Heliopan ® MA | −0.1314 | −0.1387 | 0.0073 |
| Neo Heliopan ® HMS | −0.1571 | −0.1335 | −0.0236 |
| Neo Heliopan ® BB | −0.1595 | −0.1397 | −0.0198 |
| Neo Heliopan ® AV | −0.1752 | −0.1267 | −0.0485 |
| Neo Heliopan ® NH 357 | −0.1922 | −0.2052 | 0.0130 |
| Amylcinnamaldehyde, | −0.1050 | −0.1034 | −0.0016 |
| alpha | | | |
| Iraldein, alpha | −0.0650 | −0.0561 | −0.0089 |
| Isoamyl salicylate | −0.0960 | −0.1124 | 0.0164 |
| Ethylene brassylate | 0.0010 | −0.0312 | 0.0322 |
| Lilial | −0.1280 | −0.1374 | 0.0094 |
| Cedrol | −0.0320 | −0.0319 | −0.0001 |
| Cedryl acetate | −0.0330 | −0.0592 | 0.0261 |
| Benzyl salicylate | −0.1300 | −0.1641 | 0.0341 |
| Hexylcinnamaldehyde, | −0.1250 | −0.1142 | −0.0108 |
| alpha | | | |
| Hercolyn D-E | −0.1352 | −0.0908 | −0.0444 |

From a list with these and further cosmetic ingredients with predicted partition parameters, the developer selects individual or two or more cosmetic ingredients which are particularly suitable within the scope of the method according to the present invention for substantive emulsion. Using these cosmetic ingredients, excellent emulsions are created which achieve superior substantivity.

### Example 2

W/O Emulsion, Substantivity on Skin (Water Resistance):

An exemplary formulation of a UV-protecting, perfumed W/O emulsion is as follows:

TABLE 4
W/O emulsion, formulation

| Part | Ingredients | | % (w/w) |
|---|---|---|---|
| A | Dehymuls PGPH (1) | Polyglyceryl-2 | 3.00 |
| | | Dipolyhydroxystearate | |
| | Monomuls 90-0-18 (1) | Glyceryl Oleate | 1.00 |
| | Zinc stearate (2) | Zinc stearate | 0.50 |
| | Myritol 318 (1) | Caprylic/Capric | 8.00 |
| | | Triglyceride | |
| | Cetiol SN (1) | Cetearyl Isononanoate | 7.00 |
| | Tegosoft TN (3) | C12-15 Alkyl Benzoate | 7.00 |
| | Copherol 1250 (1) | Tocopheryl Acetate | 1.00 |
| | Permulgin 2550 (4) | Beeswax (Cera Alba) | 1.20 |
| | Neo Heliopan ® AV (5) | Ethylhexyl | 1.00 |
| | | methoxycinnamate | |
| | Neo Heliopan ® E1000 (5) | Isoamyl | 1.00 |
| | | p-Methoxycinnamate | |
| | Neo Heliopan ® OS (5) | 2-Ethylhexyl salicylate | 1.00 |
| | Neo Heliopan ® HMS (5) | Homosalate | 1.00 |
| | Neo Heliopan ® MA (5) | Menthyl anthranilate | 1.00 |
| | Neo Heliopan ® MBC (5) | 4-Methylbenzylidene | 1.00 |
| | | Camphor | |
| | Neo Heliopan ® BB (5) | Benzophenone-3 | 1.00 |
| | Neo Heliopan ® 357 (5) | | 1.00 |
| B | Water, dist. | Water (Aqua) | 53.80 |
| | Glycerol 99% | Glycerol | 4.00 |
| | Phenonip (6) | | 0.50 |
| D | Fragrance (5) | Fragrance | 2.00 |

Suppliers:
(1) Cognis Deutschland GmbH, D-40191 Düsseldorf, Germany
(2) Bärlöcher, Chemische Werke, D-80992 München, Germany
(3) Th. Goldschmidt AG, D-45127 Essen, Germany
(4) Koster Keunen, bv. 5530 AB, Bladel, Netherlands
(5) Haarmann & Reimer GmbH, D-37603 Holzminden, Germany
(6) Nipa, Laboratories Ltd., CF38 2SN South Wales, UK

The example reflects the substantivity of a sunscreen emulsion. The emulsion is applied to a skin model (sheep shorn wool, because of the content of wool grease), and the substantivity thereto is tested using a watering test. The emulsion is prepared in the generally customary manner. The UV filters and a mixture of 39 odorants are incorporated in accordance with the formulations. 0.10 g of this emulsion is applied to a section of the skin model 5×10 cm in size. After drying for 30 min, the sample is stirred for 20 min in 4.5 l of water at room temperature. The sample is left to dry for 12 hours and then boiled for 10 min with 60 ml of isopropanol in a closed system. The extract is transferred to a measuring flask and made up to 100 ml. 0.10 g of the emulsion is applied to another section of the skin model. After drying for 12 hours, it is extracted with isopropanol as above. The extracts are injected in equal amounts one after the other into a GC injector, and gas chromatograms are recorded. The amounts of the UV filters and odorants found in the extracts are compared with one another (relative partition parameters). The analytical results are then used mathematically as described above.

Correlation with clogP: r²=0.097, F Test=1.07, XVr²=−0.33, outliers: 0 of 22.

Correlation with b.p.: r²=0.14, F Test=1.67, XVr²=−0.08, outliers: 0 of 22.

Correlation with b.p. and clogP: r²=0.19, F Test=1.07, XVr²=−0.49, outliers: 0 of 22.

COSMO-RS correlation: r²=0.72, F Test=10.9, XVr²=0.55, with descriptors: M₂^{X}, M₄^{X}, f_{acc}, f_{don}, outliers: 0 of 22.

TABLE 5
Example logarithmic partition parameters for substantivity on
a skin model

| Substance | Activity | Prediction | Differences |
|---|---|---|---|
| Neo Heliopan ® OS | −0.2497 | −0.2616 | 0.0119 |
| Neo Heliopan ® NH 357 | −0.1879 | −0.2198 | 0.0319 |
| Neo Heliopan ® MBC | −0.2018 | −0.1739 | −0.0279 |
| Neo Heliopan ® MA | −0.2300 | −0.2132 | −0.0169 |
| Neo Heliopan ® HMS | −0.2733 | −0.2771 | 0.0038 |
| Neo Heliopan ® E1000 | −0.2128 | −0.1501 | −0.0626 |
| Neo Heliopan ® BB | −0.2689 | −0.2375 | −0.0314 |
| Neo Heliopan ® AV | −0.2147 | −0.1280 | −0.0867 |
| Lilial | −0.2120 | −0.2200 | 0.0080 |
| Isoamyl salicylate | −0.2740 | −0.2517 | −0.0223 |
| Iraldein, alpha | −0.0470 | −0.0413 | −0.0057 |
| Ionone, beta | 0.1556 | 0.0232 | 0.1324 |
| Ionone alpha | −0.0120 | −0.0163 | 0.0043 |
| Hexylcinnamaldehyde, | −0.1399 | −0.1797 | 0.0397 |
| alpha | | | |
| Hercolyn D-E | −0.2490 | −0.1373 | −0.1117 |
| Ethylene brassylate | −0.0476 | −0.0553 | 0.0077 |
| Cedryl acetate | −0.0170 | −0.1112 | 0.0942 |
| Cedrol | −0.0111 | 0.0439 | −0.0551 |
| Camphene | −0.2420 | −0.2331 | −0.0089 |
| Benzyl salicylate | −0.1480 | −0.2797 | 0.1316 |
| Amylcinnamaldehyde, | −0.1890 | −0.1769 | −0.0121 |
| alpha | | | |
| alpha-Pinene | −0.2470 | −0.2228 | −0.0242 |

From a list with these and further cosmetic ingredients with predicted partition parameters, the developer selects individual or two or more cosmetic ingredients which are particularly suitable within the scope of the method according to the invention for substantive emulsion. Using these cosmetic ingredients, excellent emulsions are created which achieve superior substantivity.

What is claimed:

1. A method of selecting a cosmetic auxiliary or two or more cosmetic auxiliaries for a cosmetic product, comprising the steps of:
a) determining a parameter for one group of cosmetic auxiliaries from the relative concentration of a cosmetic auxiliary in the phase to be cared for relative to the concentration in the cosmetic phase,
b) determining the descriptors of cosmetic auxiliaries using a mathematical method,
c) inputting the parameters determined in the first step into a determination model and carrying out a regression calculation,
d) making a prediction for all calculated cosmetic auxiliaries based on the regression calculation,
e) predicting the cosmetic auxiliaries most effective for the manufacture of the cosmetic product.

2. A method according to claim 1, wherein the determination of the relative distribution of cosmetic auxiliaries is carried out by analysis of the concentration in the cosmetic phase and the phase to be cared for.

3. A method according to claim 1, wherein a partition equilibrium between the gas phase and a liquid phase is determined.

4. A method according to claim 1, wherein a partition equilibrium between two liquid phases is determined.

5. A method according to claim 1, wherein a partition equilibrium between the gas phase and a solid phase is determined.

6. A method according to claim 1, wherein a partition equilibrium between a liquid phase and a solid phase is determined.

7. A method according to claim 1, wherein the group of cosmetic auxiliaries comprises 2 to 100 individual compounds.

8. A method according to claim 1, wherein the group of cosmetic auxiliaries comprises 5 to 50 individual compounds.

9. A method according to claim 8, wherein the group of cosmetic auxiliaries comprises 10 to 30 individual compounds.

10. A method according to claim 1, wherein, in the calculation of the descriptors of the cosmetic auxiliaries using a mathematical method,
a) conformers are first generated,
b) then the field of force is optimized,
c) then conformers are selected by accumulation analysis,
d) then a semi-empirical structure optimization takes place,
e) then further conformers are selected by accumulation analysis,
f) then the structure is optimized using ab-initio or DFT calculations, and
g) finally, a COSMO-RS calculation is carried out.

11. A method according to claim 10, wherein a dielectric continuum calculating method is used to calculate descriptors of the cosmetic auxiliaries.

12. A method according to claim 10, wherein a mathematical determination model for the distribution between the gas phase and a liquid or solid phase is described by the function
$$\begin{matrix}log {P}_{gas , S}^{X} = {C}_{gen} ( {μ}_{gas}^{X} - {μ}_{S}^{X} ) + const . \\ = {C}_{gen} {μ}_{gas}^{X} + {C}_{S}^{0} {M}_{0}^{X} + {C}_{S}^{2} {M}_{2}^{X} + {C}_{S}^{3} {M}_{3}^{X} + {C}_{S}^{4} {M}_{4}^{X} + \\ {C}_{S}^{acc} {M}_{acc}^{X} + {C}_{S}^{don} {M}_{don}^{X} + const .\end{matrix}$$
in which the symbols have the following meanings:
P^{X}_{gas,S}: partition parameter between gas phase and liquid or solid phase;
c_{gen}: general, customized preliminary factor,
μ^{X}_{gas}: chemical potential of substance X in the gas phase according to COSMO-RS;
μ_{S}^{X}: chemical potential of substance X in the solid or liquid phase from regression; const: general regression constant;
c_{S}^{l}: expansion coefficient of the Taylor series from regression;
acc: hydrogen bridge acceptor; don: hydrogen bridge donor; and
Mᵢ^{X}: σ-moment of the i-th order of the substance X.

13. A method according to claim 10, wherein a mathematical determination model for the distribution between a liquid or solid phase on the one hand and a liquid or solid phase on the other hand is described by the function
$$\begin{matrix}log {P}_{S , {S}^{′}}^{X} = {c}_{gen} ( {μ}_{S}^{X} - {μ}_{{S}^{′}}^{X} ) + const . \\ = {c}_{S , {S}^{′}}^{0} {M}_{0}^{X} + {c}_{S , {S}^{′}}^{2} {M}_{2}^{X} + {c}_{S , {S}^{′}}^{3} {M}_{3}^{X} + {c}_{S , {S}^{′}}^{4} {M}_{4}^{X} + \\ {c}_{S , {S}^{′}}^{acc} {M}_{acc}^{X} + {c}_{S , {S}^{′}}^{don} {M}_{don}^{X} + const .\end{matrix}$$
in which the symbols have the following meanings:
P^{X}_{S,S′}: partition parameter between liquid phase S and liquid or solid phase S′;
c_{gen}: general, customized preliminary factor;
μ^{X}_{S}: chemical potential of substance X in the liquid phase S according to COSMO-RS;
μ^{X}_{S}: chemical potential of substance X in the solid or liquid phase S′ from regression;
const: general regression constant;
c_{S}ⁱ: expansion coefficient of the Taylor series from regression;
acc: hydrogen bridge acceptor;
don: hydrogen bridge donor; and
Mᵢ^{X}: σ-moment of the i-th order of the substance X.

14. A method according to claim 12, wherein a mathematical determination model is created using the σ-moments M₀^{X}, M₂^{X}, M₃^{X}, M₄^{X}, and M_{acc}^{X}, M_{don}^{X} and μ_{gas}^{X} as descriptors and a constant.

15. A method according to claim 12, wherein a mathematical determination model is created using the σ-moments M₀^{X}, M₂^{X}, M₃^{X}, M₄^{X}, and M_{acc}^{X}, M_{don}^{X} and μ_{gas}^{X} as descriptors and a constant in combination with descriptors already known.

16. A method according to claim 13, wherein a mathematical determination model is created using the σ-moments M₀^{X}, M₂^{X}, M₃^{X}, M₄^{X}, and M_{acc}^{X}, M_{don}^{X} and μ_{gas}^{X} as descriptors and a constant.

17. A method according to claim 13, wherein a mathematical determination model is created using the σ-moments M₀^{X}, M₂^{X}, M₃^{X}, M₄^{X}, and M_{acc}^{X}, M_{don}^{X} and μ_{gas}^{X} as descriptors and a constant in combination with descriptors already known.

18. A method according to claim 1, wherein a regression calculation is carried out to correlate the descriptors with the partition parameters of the cosmetic auxiliaries.

19. A method according to claim 1, wherein a prediction is made for the partition parameters of cosmetic auxiliaries.

20. A method according to claim 1, wherein the prediction of the Partition parameters of cosmetic auxiliaries is used for the manufacture of cosmetic products.

21. A method according to claim 1, wherein the cosmetic products are consumer products.

22. A method according to claim 1, wherein the cosmetic products are detergents, care compositions, air fresheners and cleaners for industrial use.

23. A method according to claim 1, wherein cosmetic products are detergents, care compositions, air fresheners and cleaners in the domestic sector.

24. A method according to claim 1, wherein cosmetic products are detergents, care compositions, air fresheners and cleaners for veterinary use.

25. A method according to claim 1, wherein cosmetic products are detergents, care compositions, air fresheners and cleaners in body hygiene.

26. Cosmetic products comprising cosmetic auxiliaries, which are selected for the cosmetic products using a mathematical determination model where
a) in a first step for one group of cosmetic auxiliaries, a parameter is determined from the relative concentration of a cosmetic auxiliary in the phase to be cared for relative to the concentration in the cosmetic phase,
b) in a second step, the descriptors of cosmetic auxiliaries are determined using a mathematical method,
c) in a third step, the parameters determined in the first step are input into a determination model and a regression calculation is carried out,
d) in a fourth step, a prediction is made for all calculated cosmetic auxiliaries based on the regression calculation,
e) in a fifth step, the cosmetic auxiliaries most effective according to the prediction are used for the manufacture of the cosmetic product.

27. Cosmetic products according to claim 26, wherein the cosmetic auxiliaries for the cosmetic products are selected using a mathematical determination model which describes the distribution of cosmetic auxiliaries between a cosmetic phase and a phase to be cared for.

28. Cosmetic products according to claim 26, wherein the cosmetic auxiliaries for the cosmetic products are selected using a mathematical determination model in which the distribution between the gas phase and a liquid or solid phase is described by the function
$$\begin{matrix}log {P}_{gas , S}^{X} = {C}_{gen} ( {μ}_{gas}^{X} - {μ}_{S}^{X} ) + const . \\ = {C}_{gen} {μ}_{gas}^{X} + {C}_{S}^{0} {M}_{0}^{X} + {C}_{S}^{2} {M}_{2}^{X} + {C}_{S}^{3} {M}_{3}^{X} + {C}_{S}^{4} {M}_{4}^{X} + \\ {C}_{S}^{acc} {M}_{acc}^{X} + {C}_{S}^{don} {M}_{don}^{X} + const .\end{matrix}$$
in which the symbols have the following meanings:
P^{X}_{gas,S}: partition parameter between gas phase and liquid or solid phase;
c_{gen}: general, customized preliminary factor;
μ^{X}_{gas}: chemical potential of substance X in the gas phase according to COSMO-RS;
μ_{S}^{X}: chemical potential of substance X in the solid or liquid phase from regression;
const: general regression constant;
c_{S}ⁱ: expansion coefficient of the Taylor series from regression;
acc: hydrogen bridge acceptor;
don: hydrogen bridge donor;
Mᵢ^{X}: σ-moment of the i-th order of the substance X.

29. Cosmetic products according to claim 26, wherein the cosmetic auxiliaries for the cosmetic products are selected using a mathematical determination model in which the distribution between a liquid or solid phase and a liquid or solid phase is described by the function
$$\begin{matrix}log {P}_{S , {S}^{′}}^{X} = {c}_{gen} ( {μ}_{S}^{X} - {μ}_{{S}^{′}}^{X} ) + const . \\ = {c}_{S , {S}^{′}}^{0} {M}_{0}^{X} + {c}_{S , {S}^{′}}^{2} {M}_{2}^{X} + {c}_{S , {S}^{′}}^{3} {M}_{3}^{X} + {c}_{S , {S}^{′}}^{4} {M}_{4}^{X} + \\ {c}_{S , {S}^{′}}^{acc} {M}_{acc}^{X} + {c}_{S , {S}^{′}}^{don} {M}_{don}^{X} + const .\end{matrix}$$
in which the symbols have the following meanings:
P^{X}_{S,S′}: partition parameter between liquid phase S and liquid or solid phase S′; c_{gen}: general, customized preliminary factor;
μ^{X}_{S}: chemical potential of substance X in the liquid phase S according to COSMO-RS;
μ^{X}_{S}: chemical potential of substance X in the solid or liquid phase S′ from regression;
const: general regression constant;
c_{S}ⁱ: expansion coefficient of the Taylor series from regression;
acc: hydrogen bridge acceptor;
don: hydrogen bridge donor; and
Mᵢ^{X}: σ-moment of the i-th order of the substance X.

30. Cosmetic products according to claim 28, wherein the cosmetic auxiliaries for the cosmetic products are selected by means of a mathematical determination model using the σ-moments M₀^{X}, M₂^{X}, M₃^{X}, M₄^{X}, and M_{acc}^{X}, M_{don}^{X} and μ_{gas}^{X} as descriptors and a constant.

31. Cosmetic products according to claim 28, wherein the cosmetic auxiliaries for the cosmetic products are selected by means of a mathematical determination model using the σ-moments M₀^{X}, M₂^{X}, M₃^{X}, M₄^{X}, and M_{acc}^{X}, M_{don}^{X} and μ_{gas}^{X} as descriptors and a constant in combination with descriptors already known.

32. Cosmetic products according to claim 29, wherein the cosmetic auxiliaries for the cosmetic products are selected by means of a mathematical determination model using the σ-moments M₀^{X}, M₂^{X}, M₃^{X}, M₄^{X}, and M_{acc}^{X}, M_{don}^{X} and μ_{gas}^{X} as descriptors and a constant.

33. Cosmetic products according to claim 29, wherein the cosmetic auxiliaries for the cosmetic products are selected by means of a mathematical determination model using the σ-moments M₀^{X}, M₂^{X}, M₃^{X}, M₄^{X}, and M_{acc}^{X}, M_{don}^{X} and μ_{gas}^{X} as descriptors and a constant in combination with descriptors already known.

34. Cosmetic products according to claim 26, wherein the cosmetic products are consumer products.

35. Cosmetic products according to claim 26, wherein the cosmetic products are detergents, care compositions, air fresheners and cleaners for industrial use.

36. Cosmetic products according to claim 26, wherein the cosmetic products are detergents, care compositions, air fresheners and cleaners in the domestic sector.

37. Cosmetic products according to claim 26, wherein the cosmetic products are detergents, care compositions, air fresheners and cleaners for veterinary use.

38. Cosmetic products according to claim 26, wherein the cosmetic products are detergents, care compositions, air fresheners and cleaners in body hygiene.

39. A method of selecting a cosmetic auxiliary or two or more cosmetic auxiliaries for the manufacturing of a cosmetic product, comprising the steps of
a) determining a parameter for one group of cosmetic auxiliaries, from the relative concentration of a cosmetic auxiliary in the phase to be cared for relative to the concentration in the cosmetic phase,
b) determining the descriptors of cosmetic auxiliaries using a mathematical method,
c) inputting the parameters determined in the first step into a determination model and carrying out a regression calculation,
d) making a prediction for all calculated cosmetic auxiliaries based on the regression calculation,
e) predicting the odorants most effective according to the prediction are used for the manufacture of the cosmetic product.

40. A method according to claim 39, wherein the determination of the relative distribution of cosmetic auxiliaries is carried out by analysis of the concentration in the cosmetic phase and the phase to be cared for.

41. A method according to claim 39, wherein the partition equilibrium between the gas phase and a liquid phase is determined.

42. A method according to claim 39, wherein the partition equilibrium between the gas phase and a solid phase is determined.

43. A method according to claim 39, wherein the partition equilibrium between a liquid phase and a solid phase is determined.

44. A method according to claim 39, wherein the partition equilibrium between two liquid phases is determined.

45. A method according to claim 39, wherein the group of cosmetic auxiliaries comprises 2 to 100 individual compounds.

46. A method according to claim 45, wherein the group of cosmetic auxiliaries comprises 5 to 50 individual compounds.

47. A method according to claim 46, wherein the group of cosmetic auxiliaries comprises 10 to 30 individual compounds.

48. A method according to claim 39, wherein, in the calculation of the descriptors of the cosmetic auxiliaries using a mathematical method,
a) first, the conformers are generated,
b) then the field of force is optimized,
c) then conformers are selected by accumulation analysis,
d) then a semi-empirical structure optimization takes place,
e) then further conformers are selected by accumulation analysis,
f) then the structure is optimized using ab-initio or DFT calculations, and
g) finally a COSMO-RS calculation is carried out.

49. A method according to claim 48, wherein a dielectric continuum calculating method is used to calculate descriptors of the cosmetic auxiliaries.

50. A method according to claims 48, wherein a mathematical determination model for the distribution between the gas phase and a liquid or solid phase is described by the function
$$\begin{matrix}log {P}_{gas , S}^{X} = {C}_{gen} ( {μ}_{gas}^{X} - {μ}_{S}^{X} ) + const . \\ = {C}_{gen} {μ}_{gas}^{X} + {C}_{S}^{0} {M}_{0}^{X} + {C}_{S}^{2} {M}_{2}^{X} + {C}_{S}^{3} {M}_{3}^{X} + {C}_{S}^{4} {M}_{4}^{X} + \\ {C}_{S}^{acc} {M}_{acc}^{X} + {C}_{S}^{don} {M}_{don}^{X} + const .\end{matrix}$$
in which the symbols have the following meanings:
P^{X}_{gas,S}: partition parameter between gas phase and liquid or solid phase; c_{gen}: general, customized preliminary factor;
μ^{X}_{gas}: chemical potential of substance X in the gas phase according to COSMO-RS;
μ_{S}^{X}: chemical potential of substance X in the solid or liquid phase from regression;
const: general regression constant;
c_{S}ⁱ: expansion coefficient of the Taylor series from regression;
acc: hydrogen bridge acceptor; don: hydrogen bridge donor; and
Mᵢ^{X}: σ-moment of the i-th order of the substance X.

51. A method according to claim 48, wherein a mathematical determination model for the distribution between a liquid or solid phase and a liquid or solid phase is described by the function
$$\begin{matrix}log {P}_{S , {S}^{′}}^{X} = {c}_{gen} ( {μ}_{S}^{X} - {μ}_{{S}^{′}}^{X} ) + const . \\ = {c}_{S , {S}^{′}}^{0} {M}_{0}^{X} + {c}_{S , {S}^{′}}^{2} {M}_{2}^{X} + {c}_{S , {S}^{′}}^{3} {M}_{3}^{X} + {c}_{S , {S}^{′}}^{4} {M}_{4}^{X} + \\ {c}_{S , {S}^{′}}^{acc} {M}_{acc}^{X} + {c}_{S , {S}^{′}}^{don} {M}_{don}^{X} + const .\end{matrix}$$
in which the symbols have the following meanings:
P^{X}_{S,S′}: partition parameter between liquid phase S and liquid or solid phase S′; c_{gen}: general, customized preliminary factor,
μ^{X}_{S}: chemical potential of substance X in the liquid phase S according to COSMO-RS;
μ^{X}_{S}: chemical potential of substance X in the solid or liquid phase S′ from regression;
const: general regression constant;
c_{S}^{l}: expansion coefficient of the Taylor series from regression;
acc: hydrogen bridge acceptor;
don: hydrogen bridge donor;
Mᵢ^{X}: σ-moment of the i-th order of the substance X.

52. A method according to claim 50, wherein a mathematical determination model is created using the σ-moments M₀^{X}, M₂^{X}, M₃^{X}, M₄^{X}, and M_{acc}^{X}, M_{don}^{X} and μ_{gas}^{X} as descriptors and a constant.

53. A method according to claim 50, wherein a mathematical determination model is created using the σ-moments M₀^{X}, M₂^{X}, M₃^{X}, M₄^{X}, and M_{acc}^{X}, M_{don}^{X} and μ_{gas}^{X} as descriptors and a constant in combination with descriptors already known.

54. A method according to claim 51, wherein a mathematical determination model is created using the σ-moments M₀^{X}, M₂^{X}, M₃^{X}, M₄^{X}, and M_{acc}^{X}, M_{don}^{X} and μ_{gas}^{X} as descriptors and a constant.

55. A method according to claim 51, wherein a mathematical determination model is created using the σ-moments M₀^{X}, M₂^{X}, M₃^{X}, M₄^{X}, and M_{acc}^{X}, M_{don}^{X} and μ_{gas}^{X} as descriptors and a constant in combination with descriptors already known.

56. A method according to claim 39, wherein a regression calculation is carried out to correlate the descriptors with the partition parameters of the cosmetic auxiliaries.

57. A method according to claim 39, wherein a prediction is made for the partition parameters of cosmetic auxiliaries.

58. A method according to claim 39, wherein the prediction of the partition parameters of cosmetic auxiliaries is used for the manufacture of cosmetic products.

59. A method for predicting solubilities of cosmetic auxiliaries in cosmetic products, comprising the steps of:
a) determining, for a group of cosmetic auxiliaries in each case individually, the solubility in the cosmetic phase,
b) determining the descriptors of cosmetic auxiliaries using a mathematical method,
c) inputting the parameters determined in the first step into a determination model and carrying out a regression calculation,
d) in a fourth step, a prediction for all calculated cosmetic auxiliaries is made based on the regression calculation, and
e) using the optimal concentration according to the prediction of the cosmetic auxiliaries for the manufacture of the cosmetic product.

60. A method according to claim 59, wherein the solubility of cosmetic auxiliaries is determined by analysis of the concentration in the cosmetic phase.

61. A method according to claim 59, wherein the solubility of cosmetic auxiliaries in a liquid phase is determined.

62. A method according to claim 59, wherein the solubility of cosmetic auxiliaries in a solid phase is determined.

63. A method according to claim 59, wherein the group of cosmetic auxiliaries comprises 2 to 100 individual compounds.

64. A method according to claim 63, wherein the group of cosmetic auxiliaries comprises 5 to 50 individual compounds.

65. A method according to claim 64, wherein the group of cosmetic auxiliaries comprises 10 to 30 individual compounds.

66. A method according to claim 59, wherein, in the calculation of the descriptors of the cosmetic auxiliaries using a mathematical method,
a) first, conformers are generated,
b) then, the field of force is optimized,
c) then, conformers are selected by accumulation analysis,
d) then, a semi-empirical structure optimization takes place,
e) then, further conformers are selected by accumulation analysis,
f) then, the structure is optimized using ab-initio or DFT calculations, and
g) finally, a COSMO-RS calculation is carried out.

67. A method according to claim 59, characterized in that a dielectric continuum calculating method is used to calculate descriptors of the cosmetic auxiliaries.
